(19) United States
(12) Patent Application Publication
(10) Pub. No.: US 2026/0354730 A1
(43) Pub. Date: Nov. 12, 2026

(54) Cardiovascular monitoring device, and related methods and systems

(71) Applicant: Vlepis Solutions PTY LTD, Sydney, New South Wales (AU)

(72) Inventors: Sharath Sriram, Windsor (AU); Madhu Bhaskaran, Windsor (AU); Rokunuzzaman Robel, Tarneit (AU); Ataur Rahman, Mont Albert North (AU); Bill Dimopoulos, Haberfield (AU)

(73) Assignee: Vlepis Solutions PTY LTD, Sydney, New South Wales (AU)

(21) Appl. No.: 19/109,220

(22) PCT Filed: Sep. 8, 2023
(86) PCT No.: PCT/AU2023/050865

§ 371 Date: Mar. 6, 2025

(30) Foreign Application Priority Data
Sep. 9, 2022 (AU) 2022902602

(51) Int. Cl. A61B 5/33; A61B 5/00; A61B 5/257; A61B 5/333

(52) U.S. Cl. CPC A61B 5/33; A61B 5/0006; A61B 5/257; A61B 5/333; A61B 2560/0204

(57) ABSTRACT

A cardiac sensor system comprises a cardiac device able to sample electrical signals produced by beating of the heart of a patient and to transmit a signal representative thereof to a receiving device capable of analysing the signals to identify features of interest and/or displaying a graphical representation of the heart beat based on the signals.

### FIELD OF THE INVENTION

The present invention relates to a patient monitoring device, in particular a cardiac monitoring device, and monitoring method.

### BACKGROUND OF THE INVENTION

Continuous and ambulatory monitoring of biopotential signals require skin-mounted electronics to be conformal; to enhance the quality of the physiological signal, and for wearer comfort.^{1,2} Typically cardiovascular monitoring requires a bulky biopotential acquisition system such as a complex wire connection and high-frequency operation to detect enough information. This in turn leads to very high-power consumption and the requirement of multifaceted signal processing methods for visual inspection. Such systems make real-time and continuous monitoring extremely challenging.^{1,3} To address these issues the assessment of cardiovascular activity of a patient has traditionally been subjective and recorded only during clinical visits.⁴⁻⁶

Unassisted and real-time monitoring of electrocardiogram (ECG) is particularly important to detect the possibility of and to diagnose and monitor various cardiovascular diseases. Surface ECG is the time-domain representation of the electrical signal of the ventricular depolarisation vector of the beating heart inside the chest.⁷ In standard cardiovascular monitoring systems this electrical signal is acquired by conventional 12 leads ECG. These systems generate 12 ECG signals, based on one reference electrode placed on the right leg (RL), three limb electrodes (left arm (LA), right arm (RA), and left leg (LL)), and six precordial or chest leads (V1-V6) placed over the torso near the heart.⁸ The limb leads demonstrate the electrical signal from the frontal plane of the heart while the chest leads represent the electrical signal from the horizontal plane of the heart.⁹

The ECG signals detected by the continuous and ambulant monitoring systems must also be capable of transmitting this information wirelessly to a receiver where a user or cardiologist can make a visual inspection. As such these ECG monitoring devices are intensely energy limited. In order to transmit the signal digitisation of the analog ECG signal is vital and requires sampling the signal with high frequency, however such systems are typically drain device power (such as a battery), hampering uninterrupted monitoring. A balance needs to be struck between a high sampling rate which allows for digitisation and spectral analysis of heart rate variability (HRV) parameters and a low rate for optimal power consumption.

There exists a need to overcome, or at least alleviate, one or more of the difficulties or deficiencies associated with the prior art.

### SUMMARY OF THE INVENTION

In one aspect, the present invention provides a device for monitoring cardiovascular activity of a patient, wherein the device includes:
a. at least one power management unit to power the device;
b. an analog frontend unit for recording cardiac data; and
c. a data transmission unit for transmission of the recorded data;
wherein the device provides a means for recording and transmission of cardiac data of the patient.

The term ‘patient’ as used herein may include, but is not limited to, an individual with cardiovascular health issues, an elderly individual, a immunocompromised individual, a health compromised individual, an individual with a suspected or undiagnosed medical condition, an athlete or any other individual wherein there is a need to measure the cardiovascular condition of said individual. For example, the ‘patient’ may be an individual who works in a high risk setting and measurement of the cardiovascular condition of said individual is required to ensure the safety of this individual. For further example, the ‘patient’ may be an individual of any age range who with a suspected or undiagnosed medical condition, wherein there is a need to measure the cardiovascular condition of said individual.

In a preferred embodiment the power management unit may include:
a. a power supply; and
b. a low dropout regulator.

In a further preferred embodiment, the device is powered by a power supply wherein the power supply may be a remote power source. In a further preferred embodiment, the device is powered by a power supply wherein the power supply may be a battery power source. In a further preferred embodiment, the power source may be a combination of a remote power source and a battery power source.

In a preferred embodiment, the device is powered remotely. In a further preferred embodiment, the device is powered by connection to a Wi-Fi, Bluetooth, cellular or low-power wide-area network power source. In a further preferred embodiment, the device includes an electromagnetic radiation receiver for receiving electromagnetic radiation and circuit for extracting power for the device from the received electromagnetic radiation. In a further preferred embodiment, the electromagnetic radiation is received from a Wi-Fi, Bluetooth, cellular or low-power wide-area network. In a further preferred embodiment, the cellular network is a 4G or 5G cellular network. In a still further preferred embodiment, the cellular network is a 6G cellular network. In an alternative preferred embodiment, the low-power wide-area network is a long range wide-area network (LoRaWAN). In a further preferred embodiment, the device is powered by any suitable low powered communication mechanisms designed for IOT (internet of things) networks. For example, the remote power source may include ISim, or on board or native connection to networks or other devices, or via cell phone connection.

In a preferred embodiment, the device is powered by a battery power source. In a particularly preferred embodiment, the battery power source is a rechargeable battery. In an alternatively preferred embodiment, the battery power source is a replaceable battery.

The battery power source as referred to herein may include a gel pack battery, lithium-ion battery, coin cell battery or any other battery type capable of powering the device for monitoring cardiovascular activity of a patient. For example, the battery type may include battery powered by tape-based power formats integrated to the device form, a battery power source from skin connectivity, or relayed from power from associated devices such as a smart watch or smartphone. The battery power source may include a lightweight biofuel cell power source. For example, the battery power source may include a paper biofuel cells, bioenzymatic fuel cell or other such lightweight, flexible, reusable, recyclable battery power source.

In a preferred embodiment, the power source is capable of powering the device for at least 8 days. The power source is preferably capable of powering the device for longer periods, for example 1 or 2 months.

For example, the battery type may include a single 145 mAh battery with a weight of 2 g which is capable of powering the device for approximately 7 days. However, by incorporating a single switching system, the device can be functional for greater periods of approximately 1 or more months.

Moreover, the firmware to run the Bluetooth Low Energy chip may be implemented to encrypt the transmitted data to avoid any cyber hazards while the data is being travelled through the cloud for remote monitoring. The optimization of minimum power dissipation, operational length, and data encryption set the platform which may include a Wireless, batteryfree, cyber-secured remote cardiovascular monitoring system.

In a preferred embodiment the analog frontend unit includes:
a. at least three electrodes;
b. integrated instrumentation amplifier; and/or
c. a second-order low pass filter.

In a further preferred embodiment, the device includes a positive terminal electrode; a negative terminal electrode; and a reference electrode. In a further preferred embodiment, each electrode includes an adhesive. The adhesive may include any suitable adhesive for applying the device and/or electrodes to the patient's skin surface. For example, the adhesive may include a soft-form adhesive to allow for movement and/or reapplication of the device or electrodes.

The electrodes of the devices described herein, may include, for example, electrodes housed within one or more film/form elements, thereby providing simplified adhesion/application of the device for the patient.

In a further preferred embodiment, the device further includes at least two resistors at the electrodes. Preferably the resistors are in the range of approximately 100 kΩ to 470 kΩ and more preferably 180 kΩ.

By the term ‘transmission unit’, as used herein, is meant any suitable element capable of transferring the recorded data to a database for storage. For example, said transmitter may include a removable SIM card, eSIM, iSIM or any suitable silicon wafer capable of transferring the recorded physiological inputs to a database for storage. In a preferred embodiment, the data transmission unit includes:
a. an analog-to-digital converter; and
b. antenna for wireless transfer of the digitised data.

In a preferred embodiment, the sampling of electrical activity is continuous or selective. In a further preferred embodiment, the sampling of electrical activity is selective.

In a preferred embodiment, the device is remotely controlled. In a further preferred embodiment, the device is activated and/or deactivated remotely. In a preferred embodiment, the electrodes are activated and/or deactivated remotely. In a further preferred embodiment, the device includes a receiver for receiving a signal for controlling a circuit to activate and/or deactivate the sensors. For example, the device may be paused, immobilised or stopped as directed by the patient, health professional or for security purposes.

In a preferred embodiment the device includes a receiver for receiving a signal for controlling a circuit to activate and/or deactivate the electrodes.

In a preferred embodiment the thickness of a substrate of the device is between approximately 80 to 120 μm to provide sufficient flexibility to allow the device to be mounted to any curved surface of human skin. In a further preferred embodiment, the thickness of the substrate of the device is between approximately 90 to 110 μm. In a further preferred embodiment, the thickness of the substrate of the device is approximately 101 μm.

In a preferred embodiment, the device includes one or more sensors or other such sensing elements capable of receiving and responding to physiological inputs. In an embodiment the physiological inputs may include a signal or stimulus emanating from the patient. For example, the signals or stimuli may include a motion sensor to monitor the movement of a patient, an electrical signal, electromagnetic radiation, a physical property, a temperature, a gas, a liquid, a protein, a hormone or other such biological factor.

In a preferred embodiment, the physiological inputs include heart rate, blood pressure, pulse, Sp02, V02 max, movement time, rest time, lay time, body temperature, electrical activity of the heart (ECG), measurement of wound recovery factors, measurement of skin surface stress factors, measurement of blood sugar levels, audio output and/or the geographic location of the patient. In a further preferred embodiment, the physiological input may include changes to red blood cell levels in the patient. In a further preferred embodiment, the physiological inputs may include changes in the patient's thoracic region, including patient mobility, movement, muscle contraction and/or strain. For example, monitoring the patient's thoracic region for said physiological inputs may be used to monitor voice detection, or for monitoring patients experiencing cardiac decline or chronic obstructive pulmonary disease (COPD).

In a further preferred embodiment, the physiological inputs may include movement rate and/or accelerometer inputs exhibited by the patient.

In a further preferred embodiment, the physiological inputs may include perfusion within the monitored region of the patient.

By ‘Sp02’, as used herein, is meant peripheral oxygen saturation, an estimation of haemoglobin oxygen saturation levels observed within the patient.

By ‘V02 max’, as used herein, is meant the maximum rate of oxygen consumption of a patient, as measured during increasing physical stress or exercise.

By the term ‘movement time’, as used herein, is meant the amount of time the patient spends moving in same movement activity.

By the term ‘rest time’, as used herein, is meant the amount of time the patient spends resting in the same rest between performing activities.

By the term ‘lay time’, as used herein, is meant the amount of time the patient spends laying down in the same laying session. The ‘lay time’ may include, but is not limited to, any length of time wherein the patient is sleeping, resting, has experienced a fall or any other incident wherein the patient is in a non-upright position.

By ‘audio output’ as used herein is meant any audible noise expressed by the patient, or noise produced by the patient. For example, audio output may include monitoring for a word count spoken by the patient. In a further example, audio output may include monitoring for patient breathing rate and/or intensity.

By ‘perfusion’ as used herein is meant the rate of passage for fluid through the patient's circulatory system or lymphatic system to an organ or a tissue.

In one embodiment, measurement of wound recovery factors includes recording levels of biological factors and/or recording physiological responses involved in wound recovery. For example, wound recovery factors of a patient recovering from surgery (outpatient) or recovering from infection may be monitored to assess for cardiac impacts such as accelerated heart rate.

Biological factors may include, but are not limited to, proteins, hormones, enzymes and/or platelets expressed as a result of wound recovery. Physiological responses may include, but are not limited to, blood clot formation, vascular constriction and/or physical contraction. In a preferred embodiment, measurement of skin surface stress factors includes recording levels of biological factors and/or recording physiological responses involved in skin surface stress recovery. For example, one such biological factor which may be monitored is hormone expression or hormone level changes within the patient, an example of one such hormone is the steroid hormone cortisol.

In a preferred embodiment measurement of blood sugar levels includes recording blood glucose levels, blood ketone levels or any other such indicator of a change in the patient's blood sugar level.

In a preferred embodiment, the device as described herein may be in part, or wholly, recyclable and/or capable of refurbishment. In a preferred embodiment, elements within the device may be replaceable. For example, elements or parts such as electrodes, battery power sources (if required), coatings, adhesive layers, films, etc. may be replaced or refurbished once the patient no longer requires use of the device. Such capability provides advantages for sustainability and/or use of devices in emerging economies where there is a need for enhanced, low cost cardiac devices.

In a second aspect there is provided a method for monitoring cardiovascular activity of a patient including:
a. applying to the patient a device for monitoring cardiovascular activity, the device including:
   i. at least one power management unit to power the device;
   ii. an analog frontend unit for recording cardiac data; and
   iii. a data transmission unit for transmission of the recorded data;
b. viewing the recorded data on a secondary device;
wherein the device includes an adhesive layer to provides a means for maintaining contact between the device and the skin surface of the patient;
wherein the device performs high-frequency sampling of cardiovascular events to achieve all the required P, Q, R, S, and T peaks of ECG signals; and
wherein the device allows for wireless communication with a secondary device.

In an embodiment, the device for monitoring cardiovascular activity may be applied to the chest or back of a patient.

In a further embodiment, the adhesive layer includes a chemical composition capable of adhering to the skin surface of the patient.

In a preferred embodiment the adhesive layer is resistant to damage from sweat and/or water. In an alternatively preferred embodiment, the adhesive layer is resistant to causing irritation or damage to the patient.

In a preferred embodiment the adhesive layer includes an adhesive selected to minimise irritation or damage to the skin of the patient, whilst maintaining functional contact with the skin for an extended period.

In a preferred embodiment the method includes use of a secondary device for receiving and/or viewing the recorded data.

In a preferred embodiment the secondary device is a remote device. In a further preferred embodiment, the secondary device is selected from a computer, smart phone, smart watch, tablet, touch screen device or any other suitable “smart device”. In a further preferred embodiment, the secondary device includes a data module configured to receive, monitor and/or analyse the cardiac data recorded by the device.

In a preferred embodiment, the secondary device is a remote device which provides a means to power the primary device remotely.

In a preferred embodiment, the device for monitoring cardiovascular activity provides real-time, continuous ECG data to body-worn electronics such as a smartphone or smart watch for the user as well as to the cloud-connected interface for the remote monitoring (i.e. by a cardiologist or other health professional). For example, the low cost, low weight (approximately 5g) device as described herein provides a means to collect data to the on-site and remotely connected interface in real-time, as well as allowing for keeping records for future use.

The device as described herein allows for use of a Bluetooth low energy system-on-chip which requires only a 50 Hz sampling frequency to detect all the critical events of the patient. Further, allowing for wireless data transmission which occurs at extremely low power (−19 dB), allowing for uninterrupted operation of the device for approximately 8 days using a simple CR1632 battery.

In a further aspect there is provided a system for monitoring cardiovascular activity of a patient including:
a. A device for monitoring cardiovascular activity of a patient, the device including:
   i. at least one power management unit to power the device;
   ii. an analog frontend unit for recording cardiac data; and
   iii. a data transmission unit for transmission of the recorded data; and
b. a secondary device for receiving the recorded data.

In a preferred embodiment the secondary device is a remote device. In a further preferred embodiment, the secondary device is selected from a computer, smart phone, tablet, touch screen device or any other suitable “smart device”. In a further preferred embodiment, the secondary device includes a data module configured to receive, monitor and/or analyse the cardiac data recorded by the device.

A ‘secondary device’, as used herein, is any such device capable of transmitting and/or receiving information for monitoring cardiovascular activity of the wearer. The secondary device may include, but is not limited to, a computer, smart phone, smart watch, tablet, touch screen device or any other suitable “smart device”.

In a preferred embodiment, the electrodes are activated and/or deactivated remotely. In a further preferred embodiment, the device includes a receiver for receiving a signal for controlling a circuit to activate and/or deactivate the electrodes.

In a fourth aspect, there is provided a wireless cardiac sensor device comprising:
a. at least three electrodes mounted on substrate for contact with skin above the heart of a patient to receive electrical signals produced by beating of the heart;
b. a digital to analogue converter for converting the signals into a digital form; and
c. a wireless transmitter of the digital form of the signals.

In a preferred embodiment, the signals from the electrodes are provided to a filter before conversion to digital form.

In a preferred embodiment, the substrate is flexible for contouring to the profile of the skin.

In a preferred embodiment, the electrodes are mounted on the substrate in a triangle shape smaller in size than the triangle formed between each of the shoulders and the midpoint between the bottoms of each side of the ribcage. In a preferred embodiment, the triangle of the electrodes is an equilateral triangle with a side length of between approximately 2-15 cm, preferably between approximately 3-12 cm and most preferably between approximately 4-10 cm in length.

In a preferred embodiment, one of the electrodes is a positive terminal electrode; one of the electrodes is a negative terminal electrode; and another of the electrodes is a reference electrode. Preferably, in use the positive terminal electrode is oriented to be closest to the right arm of the patient, the negative terminal electrode is oriented to be closest to the left arm of the patient, and the reference electrode is oriented to be closest to the left leg of the patient.

In a fifth aspect, there is provided a wireless cardiac sensor monitoring system comprising: a monitoring device comprising:
a. at least three electrodes mounted on substrate for contact with the skin above the heart of a patient to receive electrical signals produced by beating of the heart;
b. a digital to analogue converter for converting the signals into a digital form; and
c. a wireless transmitter of the digital form of the signals; and
   a receiver of the transmitted signals configured to analyse the signals to identify features of interest and/or to display a graphical representation of the heart beat based on the signals.

In a sixth aspect there is provided a process for monitoring a patient, wherein said process comprises providing a wireless cardiac sensor device, as described herein:
a. contacting at least three electrodes mounted on substrate for contact with the skin above the heart of a patient to receive electrical signals produced by beating of the heart;
b. converting the signals into a digital form; and
c. wirelessly transmitting the digital form of the signals.

In a seventh aspect there is provided a process for monitoring a patient, wherein said process comprises providing a wireless cardiac sensor device, as described herein:
a. contacting at least three electrodes mounted on substrate with skin above the heart of a patient to receive electrical signals produced by beating of the heart;
b. converting the signals into a digital form;
c. wirelessly transmitting the digital form of the signals;
d. receiving the transmitted signals; and
e. analysing the signals to identify features of interest and/or displaying a graphical representation of a trace of the heart beat based on the signals.
In a preferred embodiment, the receiving of the electrical signals (sampling period) occurs between approximately every 18-30 ms, preferably between approximately 19-25 ms, and most preferably between approximately 20-22 ms.

In a preferred embodiment, the identified features of interest comprise P, Q, R, S and/or T peaks. Preferably, the identified features of interest comprise RRI, PRI, QTI and/or QRSI.

In a preferred embodiment, the wireless transmission is of sufficient power to be received at a distance of at least approximately 0.5 to 10 m, preferably at least approximately 0.75 to 5 m, most preferably at least approximately 1 to 3 m.

In a further embodiment, the device as described herein may include further Bluetooth module(s) for sensor design improvement to accommodate additional sensors and performance.

In a further embodiment, the device as described herein may include additional Bluetooth module(s) to connect to falls gateway.
In a further embodiment, the device as described herein may include one or more enhanced BTLE Chips for connection to BTLE, Wi-Fi Hub and/or Falls/Motion IoT Gateways.

In a further embodiment, the device as described herein may include future processing on board capability for efficiency.

In a further embodiment, the device as described herein may include one or more flat, stick-able eco-cell and rechargeable battery.

In a further embodiment, the device as described herein may include one or more antennae for power harvesting to provide longer life for the device or back up power access, as required.

In a further embodiment, the device as described herein may include one or more antenna for harvesting power for device for backup power, wherein said harvesting may occur via BTLE Hub or Falls/Motion IoT Gateway.

In a further embodiment, the device as described herein may include a single (3 point) electrode wherein the electrode may be triangular in form, or alternatively a standard 3 clip electrode, for sampling.

In a further embodiment, the device as described herein may include artificial intelligence (AI) capabilities on the device or systems supporting said device. For example, said AI capabilities may provide a means for creating local alerts and processing of data recorded by the device.

For example, the device as described herein may provide for human or AI intervention to turn sensors on or off, as required. For further example, the device may provide for simultaneous or parallel processing from sensors via algorithms, including multimodal mode or advanced alerts confirming deterioration of the patient condition. In a further preferred embodiment, the device as described herein may include one or more elements which provide a means for introducing additional sensing technologies on the device to monitor alternate risks relating to cardiac impacts. For example, the device may include one or more of an accelerometer, or elements to measure Chronic Obstructive Pulmonary Disease (CPOD), Blood pressure analysis, GAIT decline via Falls/Risks Monitoring Gateway.

In a further preferred embodiment, the device as described herein may include elements which provide a means for connect to other Bluetooth gateways, potential LiFi (i.e. data transmission via lighting, in particular LED or infrared lighting which has WiFi enabled capabilities) and/or other connectivity pathways. For example, the device as described herein may allow for connection or communication with any sensor or radar of a secondary device, including secondary devices which may be mounted into walls, rooms or furniture or within close proximity to the patient for further data capture.

In an eighth aspect there is provided, a device for monitoring cardiovascular activity of a patient, wherein the device includes:
a. at least one power management unit to power the device;
b. an analog frontend unit for recording cardiac data; and
c. a data transmission unit for transmission of the recorded data;
wherein the device provides a means for recording and transmission of the cardiac data of the patient; and
wherein the device includes one or more of the following elements:
   i. a power supply selected from an additional or remote power source and/or a battery power source;
   ii. a single (3 point) electrode for sampling;
   iii. an antenna for power harvesting to provide longer life for the device and/or a back-up power access;
   iv. a means for wireless communication with a secondary device,
   v. a means connecting to BTLE, Wi-Fi Hub and/or Falls/Motion IoT Gateways

In a further preferred embodiment, the device may include a battery power source including a flat, stick-able eco-cell and/or rechargeable battery power source.

In a further preferred embodiment, the device may include an antenna for power harvesting, wherein the power harvesting may occur via BTLE Hub or Falls/Motion IoT Gateway.

In a further preferred embodiment, the device may include a means for monitoring falls detection of a patient.

In a further preferred embodiment, the device may include a single (3 point) electrode which is triangular in form.

In a further preferred embodiment, the device may include a means for wireless communication with a secondary device which allows for continuous communication with said device in real-time.

In this specification, the term ‘comprises’ and its variants are not intended to exclude the presence of other integers, components or steps.

In this specification, reference to any prior art in the specification is not and should not be taken as an acknowledgement or any form of suggestion that this prior art forms part of the common general knowledge in Australia or any other jurisdiction or that this prior art could reasonably expected to be combined by a person skilled in the art.

The present invention will now be more fully described with reference to the accompanying Examples and drawings. It should be understood, however, that the description following is illustrative only and should not be taken in any way as a restriction on the generality of the invention described above.

### BRIEF DESCRIPTION OF THE DRAWINGS/FIGURES

FIG. 1. Data flow from the biosignals to the IoT cloud and smartphone as user interface. FIG. 2. 3-Lead ECG wearable sensor patch fabrication. (a) Schematic diagram for circuit operation, showing power management, analog front end for cardiac monitoring and wireless data transmission and demonstration. (b) Schematic of the circuit design. (c) Schematic of the designed circuit in Altium. (d) Optical photograph of the fabricated device. (e) Mounted Wireless Sensor Patch WSP on skin.

FIG. 3. Power management. (a) Effect of transmission power on operational distance. (b) Effect of transmission power on battery longevity. (c) Effect of sampling rate on battery longevity.

FIG. 4. Effect of sampling rate on acquiring required peaks. Recorded signals for analog to digital conversion at sampling rates of (a) 40 ms, (b) 30 ms, (c) 20 ms, and (d) 15 ms.

FIG. 5. Validation of shrunk Einthoven triangle. (a) Placement of triangle using extended wires for T1 and T2, whereas T3 placement is based on wireless connection as shown in FIG. 2e. Recorded signals for (b) T1, (c) T2, and (d) T3. Legend: RRI is R-peak-to-R-peak Interval; PRI is P-peak-to-R-peak interval; QTI is Q-peak-to-T-peak interval; and QRSI is Q, R, and S peaks occurrence interval.

The comparative testing of the 3 leads ECG system compared to the 12 Leads commercially available devices exhibits the similar property of the frontal plane of hearts (demonstrated by Lead I, Lead II, and Lead III of 12 leads) with respect to Heart rate variability (HRV), PR intervals, QRS complex, QT, and average RR.

FIG. 6. Real-time monitoring and data analysis. (a) Screenshot of mobile screen, showing summary of ECG monitoring of the patient. (b) The overlapping of R-Wave fiducial points. (c) Commercial 12-lead ECG system.

FIG. 7. Placement of the device on a patient.

FIG. 8. Atrial depolarization, Ventricular depolarization, and ventricular repolarization including the clinically relevant P, Q, R, S, and T peaks.

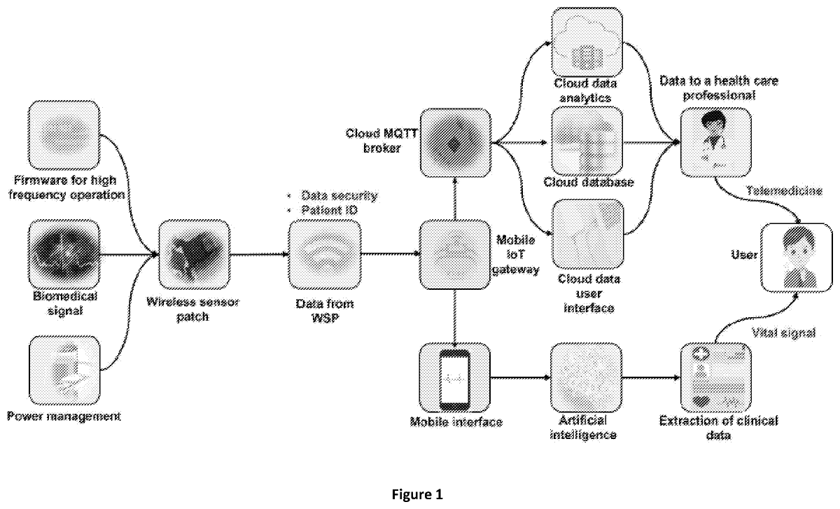

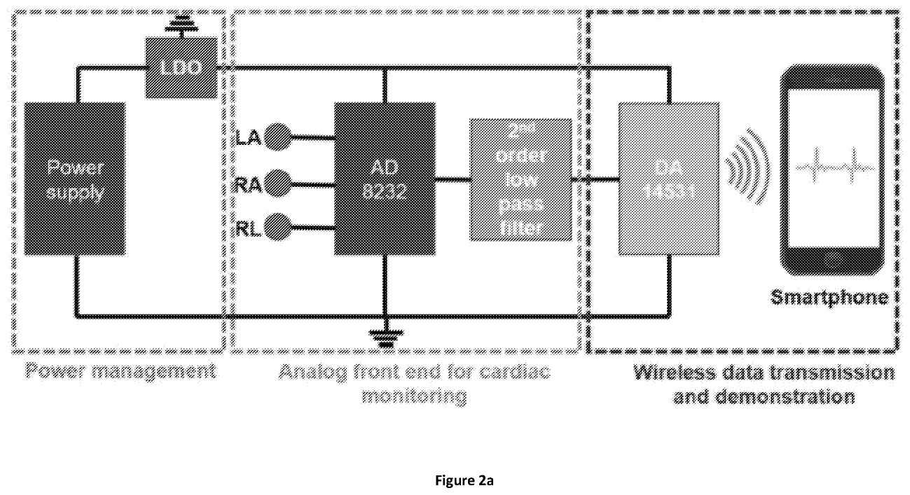

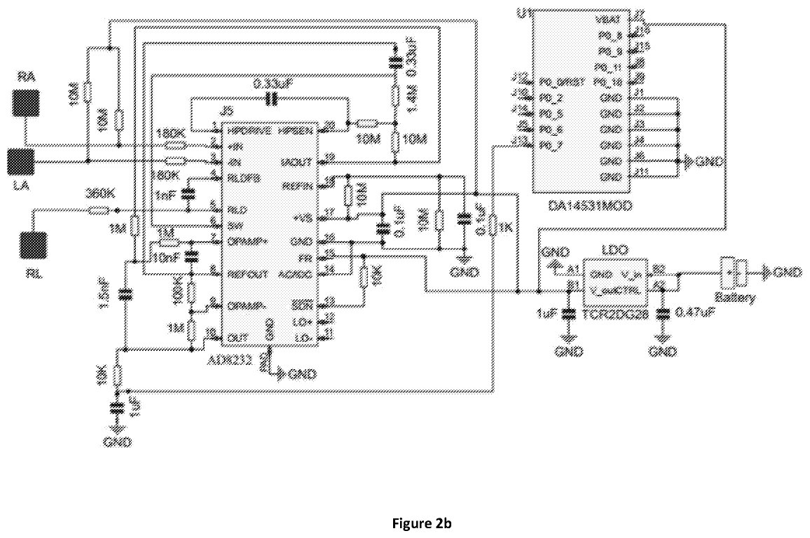

FIG. 9. Describes an alternative form of the wearable device, including the features of an Adhesion Gel, Top protection layer, Flexible Printed Circuit Board (PCBA) which includes:
9-A. A standard small cell battery that may also be replaced with a single stick-able square or triangular eco cell form battery. These features provide sufficient power to drive multi-sensing ECG, accelerometer BTLE module, iSim Module and other new and additional sensors. (eg Blood Pressure, or Oxymeter (Spo2) via additional photonics; and
9-B. A wearable device including a single (3point) electrode or alternatively a standard 3 clip electrode for sampling, wherein the electrode may be triangular in form, and may also be re-chargeable.

FIG. 10. Describes an alternative arrangement of the ECG and wearable sensor device features, including a BLE chip, ECG chip, battery holder, Copper circuits, polyimide base.

FIG. 11. Depiction of the sensor device as described herein, in use with a patient.

FIG. 12. Describes data flow from the bio-signals to the IoT cloud and falls Gateway as the access interface, including connecting to the Falls Risk Gateway or Complimentary Sensing Hub Variation of prior data and operations pathway. Further including features of connection via Bluetooth connector module/arial on patch connect and receipt on falls gateway, auto-connect function and ID detection, and secure data transfer.

FIG. 13. Integrated screening & detection process including the sensor device (new patch/new ECG patch) incorporating falls detection via Falls Detection Gateway.

FIG. 14. An alternative arrangement of the ECG and sensor device, including primary housing/soft form covers, liquid silicon rubber (LSR) or similar silicon layer, polyimide PCB surface, and a bottom adhesive layer.

FIG. 15. Describes a stepwise description of applying the wearable sensor device to a patient, including removal of underside peel protectors. Step 1, peel protectors without removing entirely. Step 2, please the adhesive onto a flat section of your upper body area and slowly peel off the protectors without stretching. Step 3, pat down the adhesive borders.

FIG. 16. An alternative arrangement of the sensor device, including ecofriendly battery design orientations in a rechargeable form which are more efficient, lower profile and suit nanotech forms, and including charge module.

### DETAILED DESCRIPTION OF THE EMBODIMENTS

Generally, a wireless cardiac sensor system comprises a wireless cardiac device able to sample electrical signals produced by beating of the heart of a patient and to transmit a signal representative thereof to a receiving device capable of analysing the signals to identify features of interest and/or displaying a graphical representation of the heart beat based on the signals.

The wireless cardiac device comprises three electrodes mounted on substrate for contact with skin above the heart of the patient to receive the electrical signals. Preferably the signals are filtered and then provides to a digital to analogue converter of the device for converting the signals into a digital form. A wireless transmitter of the device transmits the digital form of the signals to the receiving device.

In a preferred form, the substrate is thin so as to be flexible for contouring to the profile of the skin. In a preferred form, the electrodes are mounted on the substrate in an equilateral triangle shape with a side length of 5-12 cm and preferably between 6-10 cm in length. Preferably a positive terminal electrode is oriented to be closest to the right arm of the patient, a negative terminal electrode is oriented to be closest to the left arm of the patient; and a reference electrode is oriented to be closest to the left leg of the patient.

In a preferred form, the wireless cardiac device is powered by a coin battery, where the power drain is small enough for the battery to have a life of at least 1 hour, by balancing sample rate of at least 25 ms, and preferably 20 ms, between samples and transmission strength sufficient to transmit the samples at least 0.5 m, preferably with a signal power of at least −20 dB.

In a preferred form the receiving device is a portable computing device configured to identify features of interest comprising one or more of P, Q, R, S and/or T peaks, RRI, PRI, QTI and/or QRSI and the receiving device is configured to display these features of interest and a trace of the heart beat based on the received signals.

### Example 1—Wireless Sensor Patch (WSP)

The working principle of an embodiment of the wireless sensor patch is schematically represented in FIG. 2a. Fully integrated three electrodes (RA, LA, and RL) ECG with analog frontend signal conditioner (such as AD 8232 with a dimension of 4 mm×4 mm) is exploited to extract small biopotential signals in the presence of noisy conditions such as motion artefacts. The signal is amplified, such as by using integrated instrumentation amplifier which amplifies the extracted signal for 1100×. A second-order low pass filter is used to remove any additional noise before the signal is fed to the analog-to-digital converter, such as that of a Bluetooth Low Energy (BLE) module, such as DA 14531. The DA 14531, a small and low power Bluetooth 5.1 system-on-chip integrated with the antenna, is utilised for the sensor's analog data digitisation with high-frequency sampling to achieve all the required P, Q, R, S, and T peaks of ECG signals and wireless communication to a receiver, such as a smartphone. The low dropout regulator (LDO) ensures that the supply never exceeds 2.8 V. FIG. 2b illustrates the entire circuit diagram of WSP. Two 180 kΩ resistors at the input electrodes 1 and 2 are used to protect the user from any faults in the patch. To obtain the ECG signal with minimal distortion, a bandpass filter is established using a 0.5 Hz two-pole high pass filter followed by a two-pole, 40 Hz, low pass filter. For continuous and real-time monitoring, the shutdown control pin (SDN) and fast restore control pin (FR) are always connected to the supply. FIG. 2c demonstrates the schematic diagram for device fabrication using Altium PCB design tool. The thickness of both the top and bottom copper (Cu) circuit layers is 18 μm, while the thickness of the flexible polyimide layer is 75 μm. The entire substrate thickness of 101 μm offers sufficient flexibility to mount the device to any curved surface of human skin. The surface mount active components and passive components with 0201 form factor are then reflow soldered as shown in FIG. 2d. FIG. 2e demonstrates the attachment of the WSP over the torso near the heart according to the rules of the Einthoven triangle based on the bipolar leads. The so called 12 lead ECG based on Wilson central terminal invented by the F. N. Wilson and further modified as augmented leads by E. Goldberger and chest leads (V1 to V6) is modified for the present invention to access the real-time and ambulatory monitoring of cardiovascular events. Negative and positive terminals of the WSP is placed on right and left sides of the heart around the 4th rib, while the reference electrode is placed in parallel to the major ventricular depolarisation of the heart which points to the left leg. The method of device fabrication and connection of ECG electrodes is described in detail in experimental section.

### Example 2—Power Management and Firmware for High Frequency Sampling

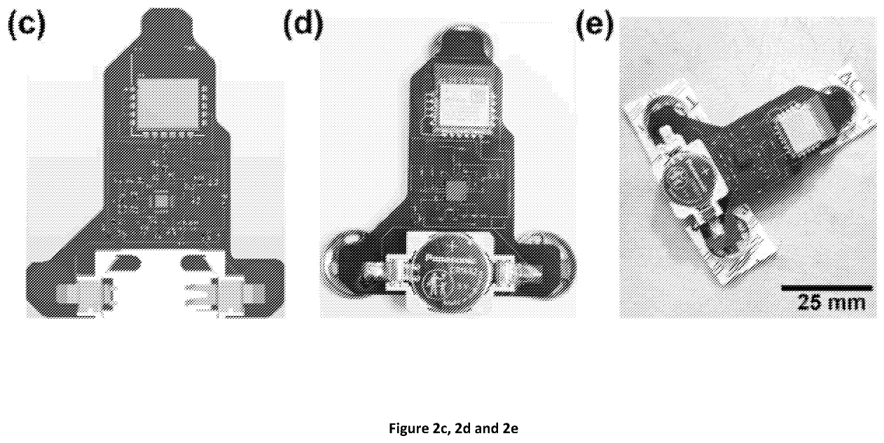

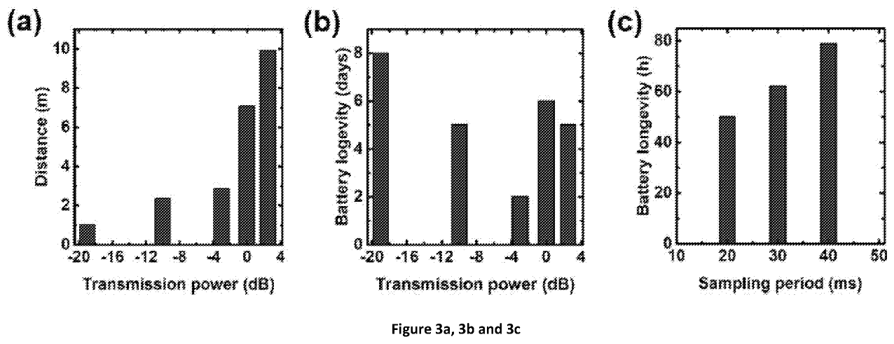

Efficient data transfer and battery longevity are the two crucial requirements for real-time and untethered cardiovascular monitoring. The Keil embedded development tool is used for modifying and developing the software development kit, SDK6.0.16.1144 for DA14531, from Dialog Semiconductor. The details of the programming and modification are discussed in the experimental section. The minimum achievable transmission power from DA14531 is −19 dB, and hence this is currently known as industry's lowest power BLE chip. The firmware is programmed using the suggested Keil IDE and flashed the different DA14531 using “Smartbond Flash Programmer” provided by Dialog Semiconductor for different transmission powers in the range from +2.5 dB to −19 dB. This enables us to understand the physical distance coverage for recording transmitted data and battery longevity. A CR1632 coin cell battery (Panasonic) of 140 mAh is used in this characterisation. FIG. 3a illustrates the distance vs. transmission power, and it can be clearly seen that distance coverage linearly increases with the transmission power. On the other hand, battery longevity decreases with the increasing transmission power as shown in FIG. 3b. Hence, there is a trade-off between distance-coverage and battery longevity. A 1 m distance is enough for ambulatory monitoring, while higher operational distance can be chosen on demand. A choice of −19 dB results in battery longevity of 8 days for advertising the availability of device connection. It is to be noted that, in this case, we observed only the advertising life for the battery not the continuous recording life on the smartphone. At the same time, sampling rate is another important factor on the battery longevity. The sampling period for 20 ms, 30 ms, and 40 ms for −19 DB transmission power on three different devices was trialled and observed was the battery power decaying for continuous recording of the signal on the smartphone screen. It can be elucidated from FIG. 3c, the battery longevity linearly decreases for the decreasing sampling period or increasing sampling frequency. Hence, it is necessary to select the appropriate sampling period not only for the battery life but also to detect all the vital peaks of ECG such as P, Q, R, S, and T as we discuss in the following paragraph.

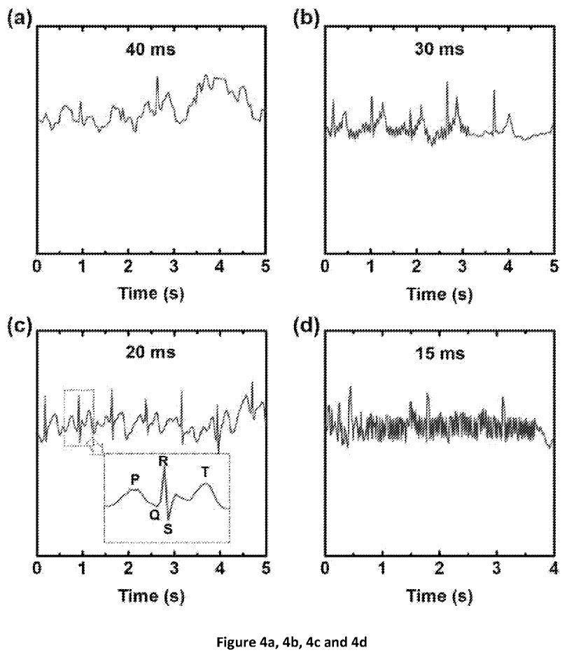

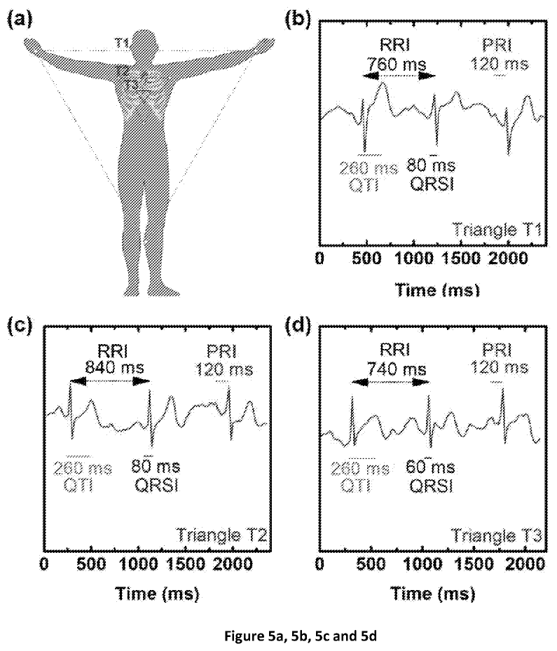

It is necessary to digitise the incoming data from the analog frontend before transmission over the air. This can be accomplished by using an integrated 4-channel 10-bit analog-to-digital converter (ADC) on the BLE system-on-chip (SoC). The tiny wearable device monitoring the ECG signal is strongly energy limited. Hence, the sampling rate of ADC needs to be selected carefully to achieve ECG signals which still enable accurate HRV analysis. A major step in analysing the HRV is to measure the time intervals between the R peaks of the ECG signals. A low sampling rate may decline the accuracy of the R-wave fiducial point. To achieve an accurate sampling period, the BLE SoC was flashed with a sampling period of 40 ms, 30 ms, 20 ms, and 15 ms. We attached the device to a volunteer chest as shown in FIG. 2e, monitored the signals on the smartphone screen, and recorded the data on a cloud-based spreadsheet. It can be clearly seen from FIG. 4a,b that the time interval between the R peaks is not the same. Moreover, there are missing R peaks for 40 ms and 30 ms sampling periods. Whereas accurate HRV is seen in FIG. 4c for a 20 ms sampling period. It is noteworthy that we rarely observe any noticeable R peaks for the 15 ms sampling period as seen in FIG. 4d. This is possibly because we have used the direct notification methodology, to overcome the necessity of utilising the SOC's internal memory. At sampling rates faster than 20 ms, due to increased packet size during each notification higher time period, clustering of sampled data might occur. Thus, we conclude that the system at 20 ms sampling period or 50 Hz sampling frequency transmits the whole ECG signal to a smartphone for visual inspection by the user or cardiologist.

### Example 3—Validation of Shrinking Triangle to be Placed Near the Torso

It is crucial to validate the presence of prominent peaks (P, Q, R, S, and T) of ECG signal as demonstrated in FIG. 4d when the WSP based on Einthoven triangle is placed on the chest. The size of the triangle can be altered to define regions (FIG. 5a). Hence, we placed the device with an extended wire connection for implementing triangles T1 and T2 as shown in FIG. 5a, whereas triangle T3 is formed just by attaching the WSP as demonstrated in FIG. 2e. T1 triangle is formed according to the 3 limb leads of 12 leads ECG system where the electrodes are placed on hand of right arm, hand of left arm, and calf of left leg. As it is necessary to shrink the triangle to realise the wearable device, we have reduced the triangle size from T1 to T2 and T3 to understand the effect of reducing size. T2 is armpit to armpit or shoulder to shoulder and down to the midway point between the bottom of each half of the ribcage. T3 is a smaller equilateral triangle of approximately 50 mm in length of each side. FIG. 5b-d demonstrate the corresponding ECG graphs obtained from different triangles. The comparison of intervals such RRI (R-peak-to-R-peak interval), PRI (P-peak-to-R-peak interval), QTI (Q-peak-to-Tpeak interval), and QRSI (intervals for QRS events) for different triangles are the key to confirm the results. It can be clearly seen from the results that PRI (120 ms), QTI (260 ms) and QRSI (80 ms) are identical for all the triangles. However, the RRI which is the major indication for heart rate (HR) is varied slightly. The RRI for TI, T2 and T3 is 760 ms (79 bpm), 840 ms (71 bpm), and 740 ms (81 bpm), respectively. Though there is a slight variation in the heart rate, it is still in the range of normal people at the resting stage. Hence, by taking all the intervals into consideration, we can confirm that the smaller triangle T3 produces the identical results of limb electrodes to infer the ECG results for the frontal plane of the heart. The results validate the performance of the functional tiny triangle vital for realising wireless sensor patches.

The comparative testing of the 3 leads ECG system compared to the 12 Leads commercially available devices exhibits the similar property of the frontal plane of hearts (demonstrated by Lead I, Lead II, and Lead III of 12 leads) with respect to Heart rate variability (HRV), PR intervals, QRS complex, QT, and average RR.

### Example 4—Real-Time Monitoring and Data Analysis

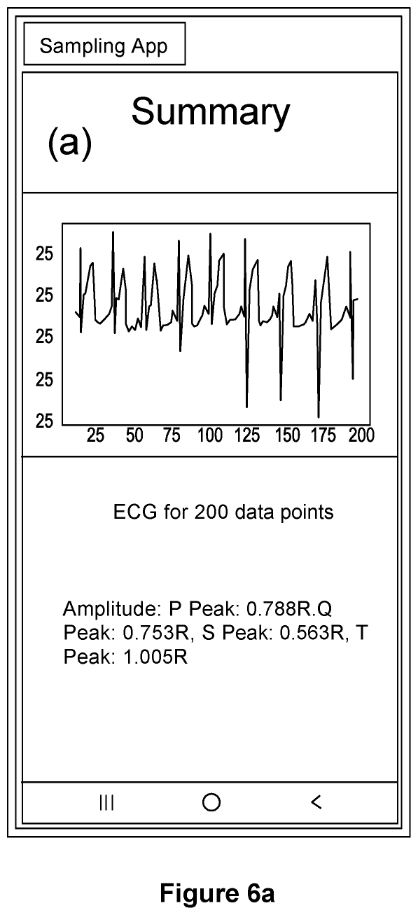

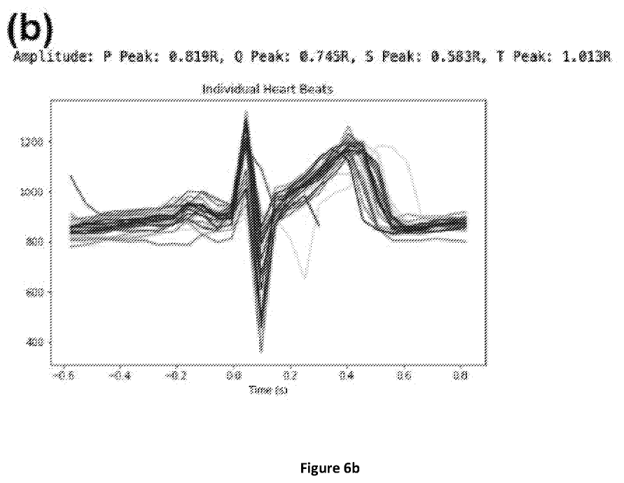

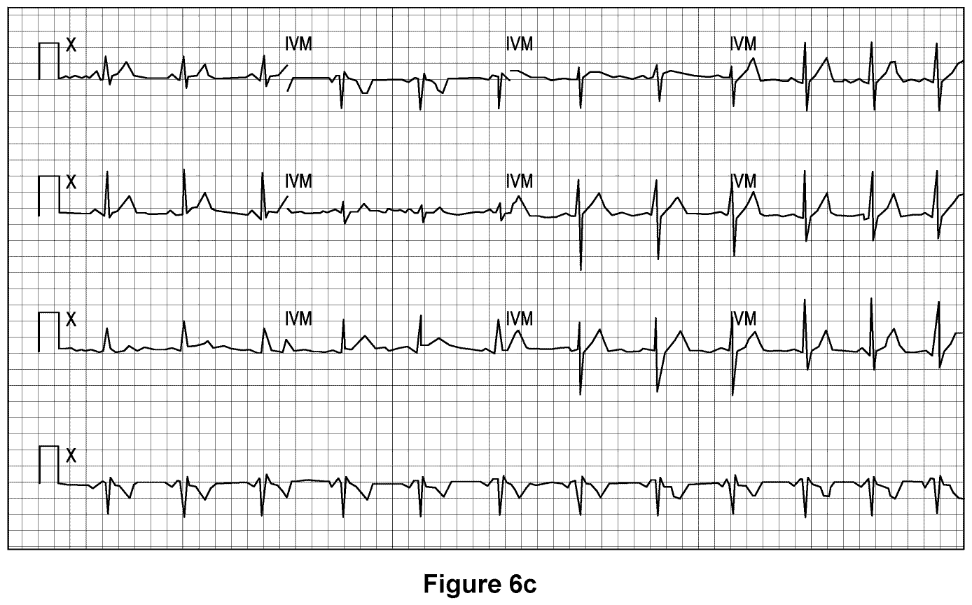

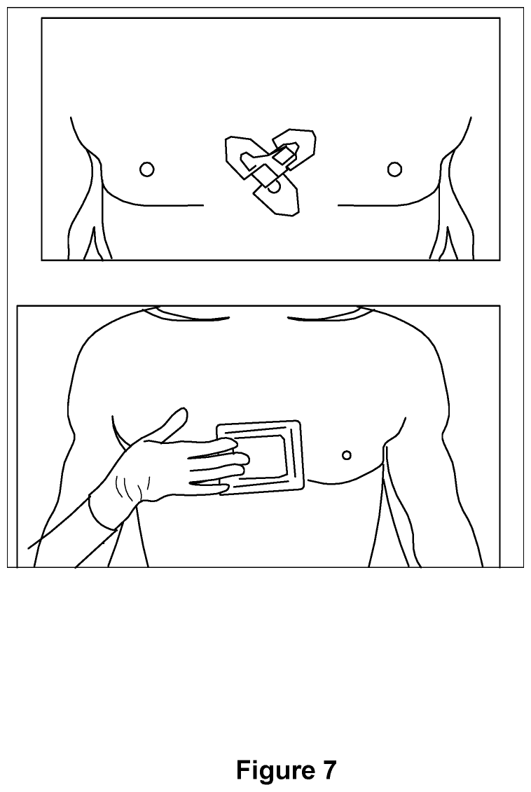

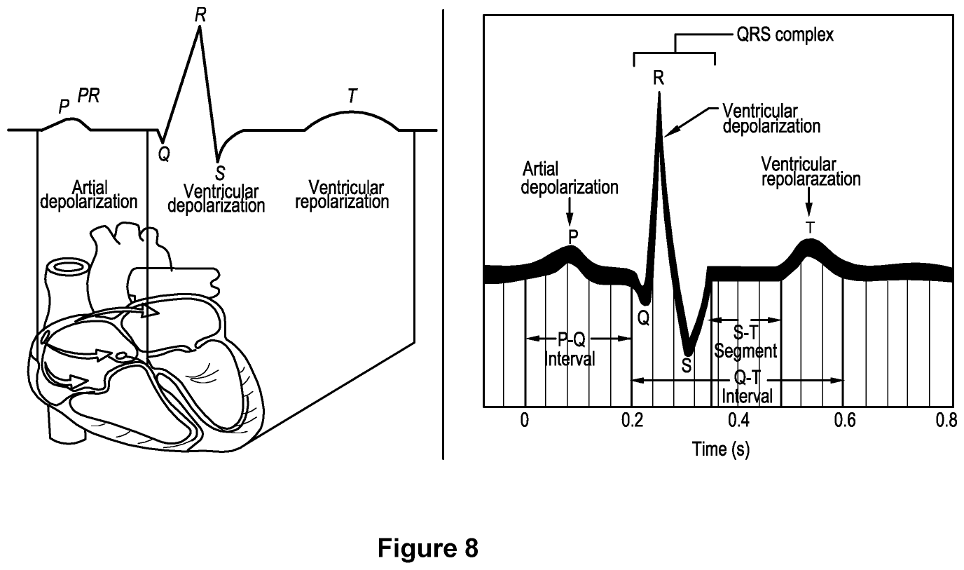

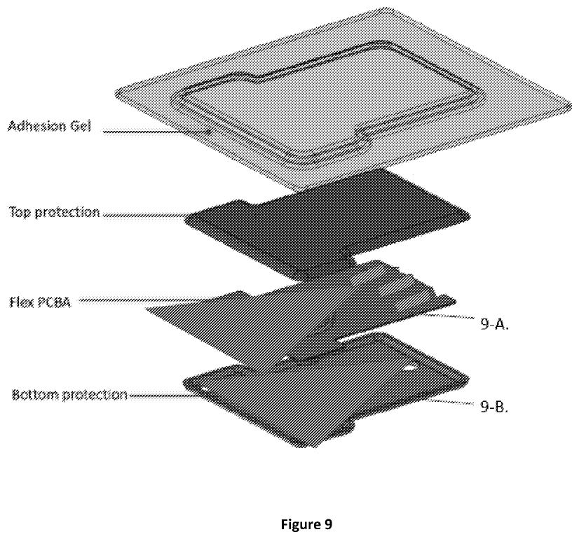

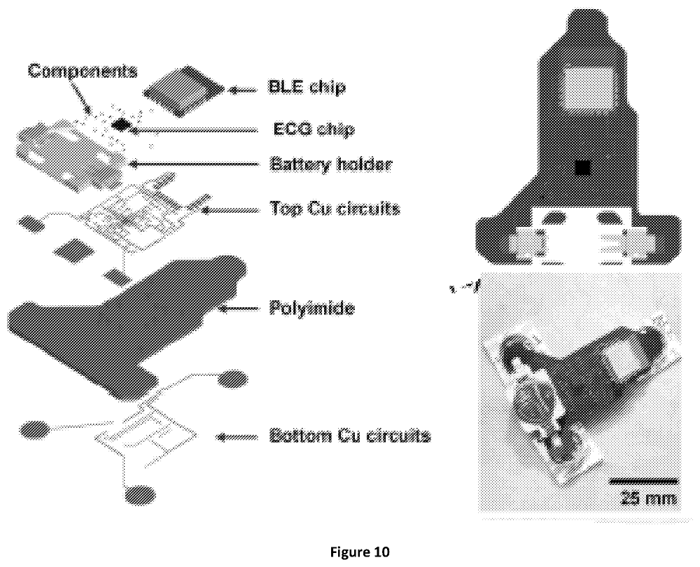

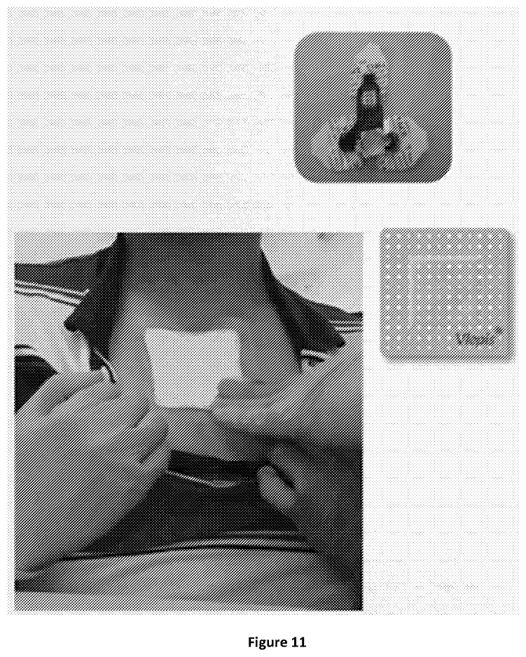

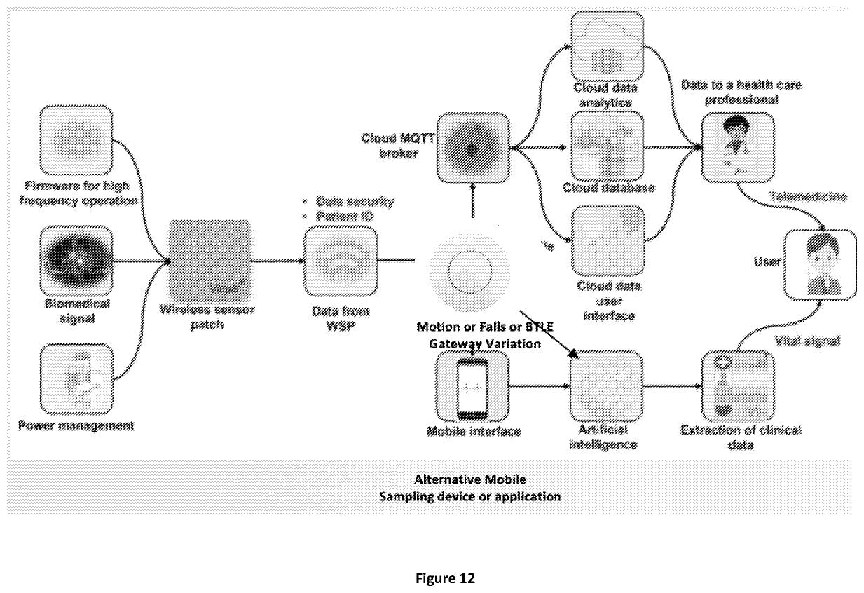

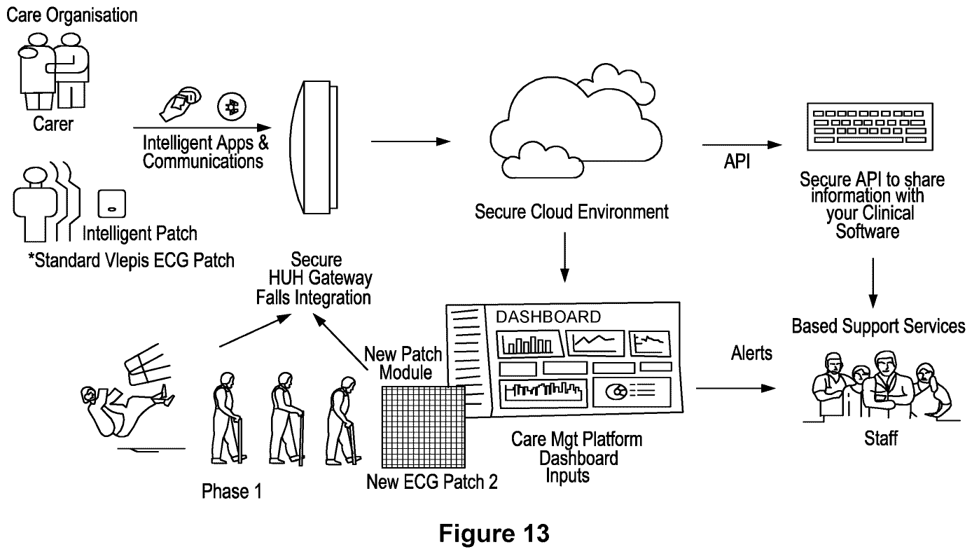

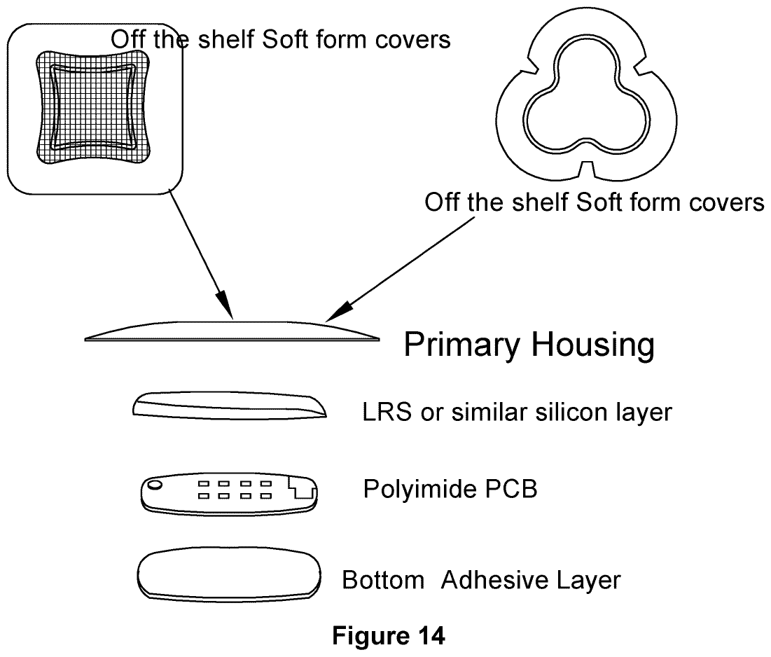

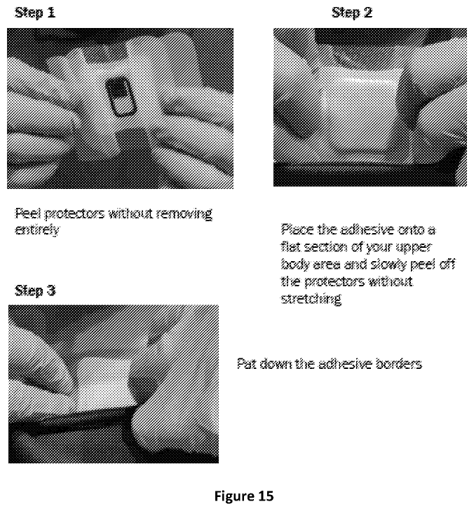

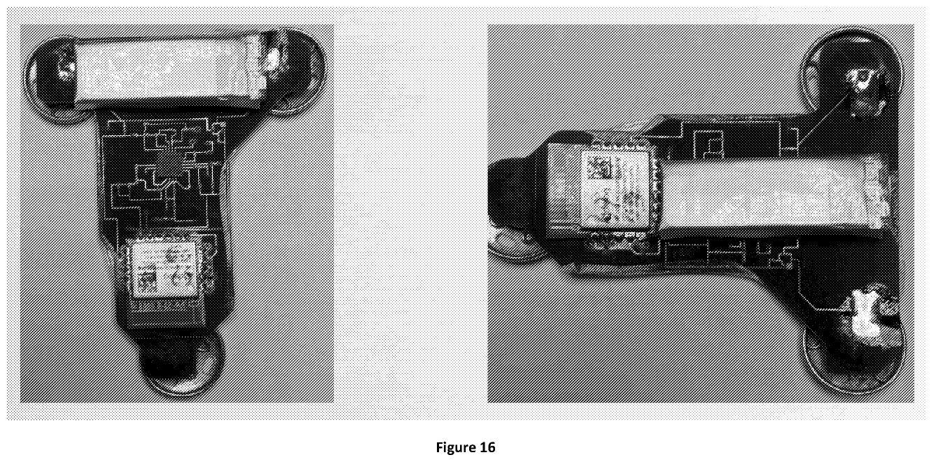

FIG. 6a demonstrates the real-time monitoring of ECG signals on the smartphone screen and cloud connected interface. The app development for smartphones and data management system development for cloud connected interface are discussed in detail in the methodology section. For the simplicity of discussion, we include the screenshots of the summary from the app and cloud connected computer in FIG. 6. In the graph, the presence of P, QRS, and T peaks, which are the vital peaks for diagnosing cardiovascular issues, are shown. The amplitude of the peaks and RR interval (RRI) on the mobile screen infer the heart rate HR and HRV which can be used for risk stratification. The overlapping of R-wave fiducial points as shown in FIG. 6b for the entire data set which can be seen on the computer screen is an important indication of any HR variation. Eventually, by obtaining the R-wave fiducial points, the RRI for each signal (800 ms) is calculated between successive R peaks of the ECG signal. It also verifies that 50 Hz sampling rate is adequate for obtaining time-domain HRV parameters with reasonable accuracy. A prolonged QT interval can predispose patients to life-threatening arrythmias. Monitoring this abnormality is difficult and usually requires 12-lead ECG system. The presence of clear T peak in our 3-lead ECG system can help to understand the QT interval. The average QT interval from the system of the present invention is 160 ms. Hyperkalaemia is a common cause of life-threatening arrythmias in patients with cardio-vascular disease, especially in individuals with underlying renal insufficiency. This disease is predicted using the QRS widening and PR shortening. The QRS interval measured in the system is 60 ms and the PR interval is 120 ms. As the system is based on bipolar leads of Einthoven triangle which are leads I, II, and III of 12-lead ECG systems, it is essential to carry out a comparative study with those 3 leads of 12-leads ECG. A Welch Allyn Connex Cardio 12-lead ECG system is used, and the result is represented in in FIG. 6c. Clearly seen is the result is quite similar as lead I and lead II. It is noteworthy to mention that the limb leads (I, II, III, aVR, aVL, and aVF) are used to achieve the ECG data of the frontal plane of the heart while the chest leads (V1-V6) are used to get the ECG data of the horizontal plane of the heart.

This wearable 3 lead ECG system which gives partial frontal data is useful for home-based monitoring to infer early any anomalies which can then be analysed further using 12 lead ECG thus significantly allowing early intervention and better cardiovascular health management.

### Example Materials and Methods

### Fabrication of Wireless, Ble Electronics

A flexible printed circuit board for the wireless BLE electronics for cardiac monitoring devices was obtained by patterning a commercially available DuPont Pyralux AP8535R substrate using an LPKF U4 UV laser system. The connection between the top and bottom circuit layers of the device was made using through-holes. The surface-mounted components, including active components such as BLE SoC (Dialog semiconductor DA14531), low power ECG frontend (Analog Devices AD 8232), LDO (Toshiba TCR2DG28LF), battery (Panasonic CR1632), and passive components such as resistors and capacitors in 0201 package were assembled using low-temperature soldering. A commercial ECG electrode (MIKROE-2456 of MikroElektronika) is used to attach the WSP to the skin as well as to match the impedance between the device electrode and skin surface to achieve a better signal.

### Firmware Development for Controlling Sampling Frequency, and Transmission Power

In this work, a Dialogue Semiconductors SmartBond TINY™ DA14531 is used, which is the BLE solution to power the IoT devices. The SoC was chosen because it is currently one of the smallest and lowest power Bluetooth 5.1 SoC. This reduces the cost of adding BLE in wearable tag-based systems.

The SoC specifications and entire details are provided in the respective companies' websites as per the reference [10], which are incorporated herein by reference.¹⁰ Further examples from the company, on utilizing the SoC are provided in the GitHub link as per the reference.[11], which are incorporated herein by reference.¹¹

The software development kit (SDK) provided by the company is very elaborate and by utilising the same, the SoC was programmed following the sleep mode tutorial,[12] which are incorporated herein by reference.¹² The SoC is configured to operate in extended sleep mode to expand the battery life. In this mode, the radio domain, all the peripheral domains, and the system domain of SoC are powered down while the systems random access memory (SRAM) is powered on to retain data. Furthermore, the SoC provides the provision to set the transmission power level from −19.5 dBm up to +2.5 dBm, which are utilised during power optimization studies discussed above. Lastly, the direct notification frequency is set, as per the sampling frequency required.

### Android App Development for Smartphone

An Android application was created to collect the data through Bluetooth low-energy protocol. The application was developed in Android Studio using Java as the primary language. A standard Android BLE protocol was established to collect the data through specific characteristics and then displayed on the screen using a graph. This data was also stored in the backend, which was later used to perform some simple analytics to extract useful information. A Neurokit24 was used for this purpose and displayed some key information. This includes finding the amplitude of the P, Q, R, S, T peaks and displaying all the collective heartbeats in a single plot to analyse the difference in heartbeats among different observations taken.

As a further example, the mobile application records and plots the ECG data on a smartphone for real-time visual monitoring and transmits the data to the cloud-connected interface for remote access by healthcare professionals. The demonstration of this wireless patch with low power operation and integration to the cloud-connected interface method provides a significant step for non-assisted home-based caring, in ambulatory settings, and for enabling preventive health management using devices without being widely constrained by battery and tethered operation.

For further example, the above described android/mobile applications may also be developed to operate on iOS devices.

### Cloud-based Data Management

The same process of data analytics is replicated through a computer as well. Since the data that is collected from the Android application is later moved online (‘the cloud’), the data is imported from the online cloud spreadsheet into a Python notebook and similar calculations performed as in the above step for the case of the Android application. Some key insights again include finding P, Q, R, S, T peaks amplitude and their intervals, displaying the heartbeat of the collective data along with some other useful information.

Finally, it is to be understood that various alterations, modifications and/or additions may be made without departing from the spirit of the present invention as outlined herein.

### REFERENCES

[1] Jeong, H. et al. Modular and Reconfigurable Wireless E-Tattoos for Personalized Sensing. Advanced Materials Technologies 4, 1900117, doi:10.1002/admt.201900117 (2019).
[2] Cosoli, G., Spinsante, S., Scardulla, F., D'Acquisto, L. & Scalise, L. Wireless ECG and cardiac monitoring systems: State of the art, available commercial devices and useful electronic components. Measurement 177, 109243, doi:10.1016/j.measurement.2021.109243 (2021).
[3] Zulqarnain, M. et al. A flexible ECG patch compatible with NFC RF communication. Npj Flexible Electronics 4, doi:10.1038/s41528-020-0077-x (2020).
[4] Bayoumy, K. et al. Smart wearable devices in cardiovascular care: where we are and how to move forward. Nat Rev Cardiol 18, 581-599, doi:10.1038/s41569-021-00522-7 (2021).
[5] Li, C. et al. Design of biodegradable, implantable devices towards clinical translation. Nature Reviews Materials 5, 61-81, doi:10.1038/s41578-019-0150-z (2019).
[6] Jayaraman, P. P., Forkan, A. R. M., Morshed, A., Haghighi, P. D. & Kang, Y. B. Healthcare 4.0: A review of frontiers in digital health. WIREs Data Mining and Knowledge Discovery 10, doi:10.1002/widm.1350 (2019).
[7] Jurak, P. et al. Novel ultra-high-frequency electrocardiogram tool for the description of the ventricular depolarization pattern before and during cardiac resynchronization. J Cardiovasc Electrophysiol 31, 300-307, doi:10.1111/jce.14299 (2020).
[8] Malmivuo, J. & Plonsey, R. Bioelectromagnetism—Principles and Applications of ioelectric and Biomagnetic Fields. (1995).
[9] Gargiulo, G. D., Bifulco, P., Cesarelli, M., Fratini, A. & Romano, M. Problems in Assessment of Novel Biopotential Front-End with Dry Electrode: A Brief Review. Machines 2, 87-98 (2014).
[10] SmartBond DA14530 and DA14531, https://www.dialogsemiconductor.com/products/bluetooth-low-energy/da14530-andda14531, (accessed on 12th of May, 2022).
[11] Dialog-semiconductor/BLE_SDK6_examples, https://github.com/dialogsemiconductor/BLE_SDK6_examples, (accessed on 12th of May, 2022)
[12] DA14531/DA14585-586 Sleep mode tutorial, http://lpccs-docs.dialog semiconductor.com/DA14531_Sleep_Mode/index.html, (accessed on 12th of May, 2022).

1-45. (canceled)

46. A device for monitoring cardiovascular activity of a patient, wherein the device includes:
a. at least one power management unit to power the device;
b. an analog frontend unit for recording cardiac data; and
c. a data transmission unit for transmission of the recorded data;
wherein the device provides a means for recording and transmission of the cardiac data of the patient.

47. A device according to claim 46, where the power management unit further includes:
a. a power supply, preferably selected from an additional or remote power source and/or a battery power source, preferably the remote power source is selected from a Wi-Fi, Bluetooth, cellular or low-power wide-area network power source; and
b. a low dropout regulator (LDO).

48. A device according to claim 47, wherein the device includes an electromagnetic radiation receiver for receiving electromagnetic radiation and circuit for extracting power for the device from the received electromagnetic radiation.

49. A device according to any one of claim 46, wherein the analog frontend unit includes:
a. at least three electrodes, preferably a positive terminal electrode; a negative terminal electrode; and a reference electrode;
b. an integrated instrumentation amplifier; and/or
c. a second-order low pass filter.

50. A device according to claim 49, wherein the device further includes at least two resistors at the electrodes.

51. A device according to claim 46, wherein the data transmission unit includes:
a. an analog-to-digital converter; and
b. antenna for wireless transfer of the digitised data.

52. A device according to claim 46, wherein the means for recording and transmission of the cardiac data of the patient includes continuous or selective sampling of electrical activity of the patient, preferably the sampling of electrical activity is selective.

53. A device according to claim 46, wherein the device is remotely controlled.

54. A device according to claim 46, wherein the device is activated and deactivated remotely, and wherein the device includes a receiver for receiving a signal for controlling a circuit to activate and/or deactivate the sensors.

55. A method for monitoring cardiovascular activity of a patient including:
a. applying to the patient a device for monitoring cardiovascular activity according to claim 46; and
b. viewing the recorded data on a secondary device;
wherein the analog frontend unit includes an adhesive layer for maintaining contact between the electrodes and the skin surface of the patient;
wherein the device performs high-frequency sampling of cardiovascular events to achieve all the required P, Q, R, S, and T peaks of ECG signals; and
wherein the device allows for wireless communication with a secondary device, preferably the secondary device is selected from a computer, smart phone, smart watch, tablet, touch screen device or any other suitable “smart device”.

56. A system for monitoring cardiovascular activity of a patient including:
a. a device for monitoring cardiovascular activity of a patient according to claim 46; and
b. a secondary device for receiving the recorded data, preferably the secondary device is selected from a computer, smart phone, smart watch, tablet, touch screen device or any other suitable “smart device”.

57. A wireless cardiac sensor device comprising:
at least three electrodes mounted on substrate for contact with skin above the heart of a patient to receive electrical signals produced by beating of the heart;
a analogue to digital converter for converting the signals into a digital form; and
a wireless transmitter of the digital form of the signals.

58. A wireless cardiac sensor device according to claim 57, wherein the signals from the electrodes are provided to a filter before conversion to digital form.

59. A wireless cardiac sensor device according to claim 57, wherein the electrodes are mounted on the substrate in a triangle shape smaller in size than the triangle formed between each of the shoulders and the midpoint between the bottoms of each side of the ribcage, preferably the triangle of the electrodes is an equilateral triangle with a side length of between approximately 2-15 cm.

60. A wireless cardiac sensor device according to claim 57, wherein one of the electrodes is a positive terminal electrode; one of the electrodes is a negative terminal electrode; and
another of the electrodes is a reference electrode, preferably in use the positive terminal electrode is oriented to be closest to the right arm of the patient.

61. A wireless cardiac sensor monitoring system comprising:
i) a monitoring device according to claim 57; and
ii) a receiver of the transmitted signals configured to analyse the signals to identify features of interest and/or to display a graphical representation of the heart beat based on the signals.

62. A wireless cardiac sensor monitoring system according to claim 61, wherein the receiver of the transmitted signals is a secondary device selected from a computer, smart phone, smart watch, tablet, touch screen device or any other suitable “smart device”.

63. A wireless cardiac sensor monitoring system according to claim 62, wherein the secondary device includes a data module configured to receive, monitor and/or analyse the cardiac data recorded by the device.

64. A process for monitoring a patient, wherein said process comprises:
a. providing a wireless cardiac sensor device according to claim 46;
b. contacting at least three electrodes mounted on the substrate with skin above the heart of a patient to receive electrical signals produced by beating of the heart;
c. converting the signals into a digital form; and
d. wirelessly transmitting the digital form of the signals.

65. A process for monitoring a patient, wherein said process comprises:
a. providing a wireless cardiac sensor device according to claim 46;
b. contacting at least three electrodes mounted on the substrate with skin above the heart of a patient to receive electrical signals produced by beating of the heart;
c. converting the signals into a digital form;
d. wirelessly transmitting the digital form of the signals, preferably the wireless transmission is of sufficient power to be received at a distance of at least approximately 0.5 to 10 m;
e. receiving the transmitted signals, preferably the receiving of the electrical signals (sampling period) occurs approximately between every 18 to 30 ms; and
f. analysing the signals to identify features of interest and/or displaying a graphical representation of a trace of the heart beat based on the signals, preferably the identified features of interest comprise P, Q, R, S and/or T peaks, RRI, PRI, QTI and/or QRSI.

66. A device for monitoring cardiovascular activity of a patient, wherein the device includes:
a. at least one power management unit to power the device;
b. an analog frontend unit for recording cardiac data; and
c. a data transmission unit for transmission of the recorded data;
wherein the device provides a means for recording and transmission of the cardiac data of the patient; and
wherein the device includes one or more of the following elements:
i. a power supply selected from an additional or remote power source and/or a battery power source;
ii. a single (3point) electrode array for sampling, preferably triangular in form;
iii. an antenna for power harvesting to provide longer life for the device and/or a back-up power access;
iv. a means for wireless communication with a secondary device; and/or
v. a means connecting to BTLE, Wi-Fi Hub and/or Falls/Motion IoT Gateways.

67. A device according to claim 66, wherein the battery power source includes a flat, stick-able eco-cell and/or rechargeable battery power source and/or power harvesting may occur via the BTLE Hub or Falls/Motion IoT Gateway.

68. A device according to claim 66, wherein the device includes means for monitoring falls detection of a patient.

69. A device according to claim 66, wherein the means for wireless communication with a secondary device allows for continuous communication with said device in real-time.
